(19) United States
(12) Patent Application Publication
(10) Pub. No.: US 2025/0302657 A1
(43) Pub. Date: Oct. 2, 2025

(54) Penis enlargement device

(71) Applicant: Baseem Najjar, Lewes, DE (US)

(72) Inventor: Baseem Najjar, Lewes, DE (US)

(21) Appl. No.: 18/618,990

(22) Filed: Mar. 27, 2024

Related U.S. Application Data
(63) Continuation-in-part of application No. 18/582,593, filed on Feb. 20, 2024.

(51) Int. Cl. A61F 5/41

(52) U.S. Cl. CPC A61F 5/41; A61F 2005/412

(57) ABSTRACT

According to an aspect of the present invention, there is provided a penis enlargement device, comprising: a base; two rods being rulers with teeth on them; a tension scale; two buttons next to the tension scale configured to release the lock on the teeth on the rods to change length; a cup configured to attach to the penis of a user; and a pump configured to create a vacuum in the cup.

### INCORPORATION BY REFERENCE

U.S. Pat. No. 11,918,505 to which the present application claims priority is hereby incorporated by reference in its entirety.

### BACKGROUND

Methods and devices for enhancing the penis of a male individual are well-known in the patent literature.

For example, U.S. Pat. No. 20,150,202,109 discloses a penis enlargement device comprising a substantially flat member with a hole and neck adapted to fit over a human penis to be positioned flat against a user's lower abdomen; a ring member also adapted to fit around the base of the penis; a waist strap attached to said flat member adapted to hold the flat member in place; a substantially cylindrical cover attached to said neck adapted to connect to either stretch cords or to a weight container; whereby, weight or force is applied to the base of the penis causing the penis to enlarge over time.

U.S. Pat. No. 7,802,577 discloses a harness for stretching the penis, includes a belt for encircling the waist of the user, with a fastening mechanism secured to a rear part of the belt. A first tractive means applies tractive forces at the base of the penis of a user of the harness, and is secured to the belt via the fastening mechanism. A second tractive means applies tractive forces at the edge of the head of the penis of a user of the harness, and is also secured to the belt via the fastening mechanism. The first tractive means and the second tractive means apply a stretching force to the penis of the user of the harness.

U.S. Pat. No. 8,075,473 discloses an extension device for permanent penis enlargement and straightening via long-term elongation, the extension device comprising: a fastening means for application to a penis, the fastening means having an accommodation body, the accommodation body: being dimensionally stable, accommodating a glans penis of the penis over the entire surface of the glans penis when the glans penis is inserted into the extension device, having an inner contour essentially corresponding to a shape of the glans penis, having an opening for allowing insertion of the glans penis, and having an inner surface; a pulling apparatus connected to the fastening means; an elastic tube seal connected to the opening of the accommodation body; and a lubricant located on the inner surface of the accommodation body; wherein the pulling apparatus is formed by: a support, strap, belt or other clothing accessory or piece of clothing, the support, strap, belt or other clothing accessory or piece of clothing being connected to the accommodation body, being held on a body of the user, and being used as a stop, a stationary object, a frame supported on a body of the user, the frame being adjustable in length, or a weight attached to the accommodation body.

Nonetheless, prior art methods and devices suffer from significant drawbacks, including physical danger and inefficiency in bringing about the desired penile enlargement.

### SUMMARY OF INVENTION

Therefore, the present invention provides an improved device for enlarging the penis of a user.

According to an aspect of the present invention, there is provided a penis enlargement device, comprising: a base; two rods being rulers with teeth on them; a tension scale; two buttons next to the tension scale configured to release the lock on the teeth on the rods to change length; a cup configured to attach to the penis of a user; and a pump configured to create a vacuum in the cup.

### BRIEF DESCRIPTION OF THE DRAWINGS

FIG. 1 illustrates a penis enlargement device according to an embodiment of the present invention.

### DETAILED DESCRIPTION

Embodiments illustrative of the present invention will be described with reference to the attached drawing.

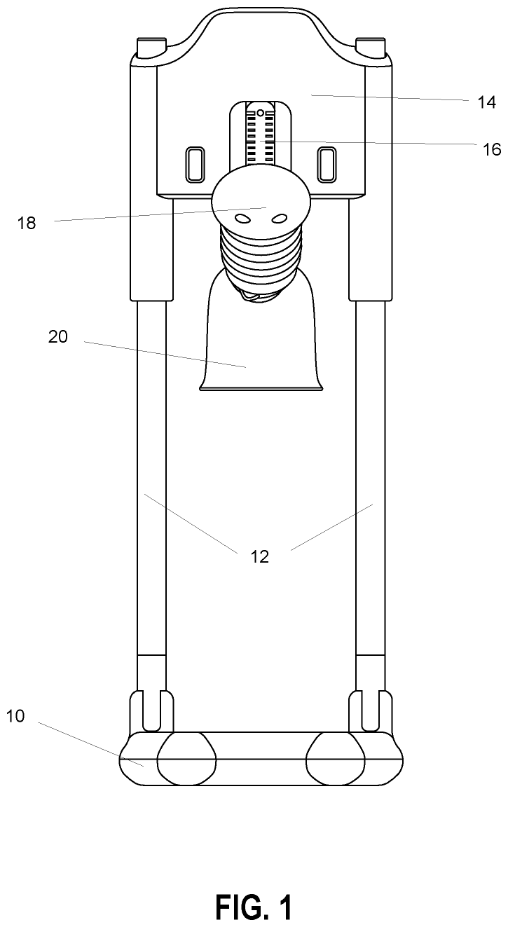

FIG. 1 illustrates a penis enlargement device according to an embodiment of the present invention.

The device has silicon base 10.

The rods 12 are rulers and they have teeth on them.

In the middle is tension scale 16.

The two buttons next to the tension scale release the lock on the teeth on the bar to change length.

It changes in 5 mm increments.

Attached to the cup 20 is a pump 18. The pump creates vacuum in the cup.

The embodiments described above are given merely for example and for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A device for enlargement of the penis of a user, comprising:
a base;
two rods being rulers with teeth on them;
a tension scale;
two buttons next to the tension scale configured to release the lock on the teeth on the rods to change length;
a cup configured to attach to the penis of a user; and
a pump configured to create a vacuum in the cup.

2. The device for enlargement of the penis of a user of claim 1, wherein the length changes in 5 mm increments.
